(19) United States
(12) Patent Application Publication
(10) Pub. No.: US 2023/0304189 A1
(43) Pub. Date: Sep. 28, 2023

(54) TCR ENGINEERING WITH DEEP REINFORCEMENT LEARNING FOR INCREASING EFFICACY AND SAFETY OF TCR-T IMMUNOTHERAPY

(71) Applicant: NEC Laboratories America, Inc., Princeton, NJ (US)

(72) Inventors: Renqiang Min, Princeton, NJ (US); Hans Peter Graf, South Amboy, NJ (US)

(21) Appl. No.: 18/174,799

(22) Filed: Feb. 27, 2023

Related U.S. Application Data
(60) Provisional application No. 63/323,540, filed on Mar. 25, 2022.

(51) Int. Cl. C40B 30/04; G06N 3/092; G06N 3/0442; C40B 20/04; G16B 40/00

(52) U.S. Cl. CPC C40B 30/04; G06N 3/092; G06N 3/0442; C40B 20/04; G16B 40/00

(57) ABSTRACT

A method for implementing deep reinforcement learning with T-cell receptor (TCR) mutation policies to generate binding TCRs for immunotherapy includes extracting peptides to identify a virus or tumor cells, collecting a library of TCRs from patients, predicting interaction scores between the extracted peptides and the TCRs from the patients, developing a deep reinforcement learning framework with TCR mutation policies to generate TCRs with maximum binding scores, defining reward functions, outputting mutated TCRs, ranking the outputted TCRs to utilize top-ranked TCR candidates to target the virus or the tumor cells, and for each top-ranked TCR candidate, repeatedly identifying a set of self-peptides that the top-ranked TCR candidate binds to and further optimizing it greedily by maximizing a sum of its interaction scores with a given set of peptide antigens while minimizing a sum of its interaction scores with the set of self-peptides until stopping criteria of efficacy and safety are met.

### RELATED APPLICATION INFORMATION

This application claims priority to Provisional Application No. 63/323,540 filed on Mar. 25, 2022, the contents of which are incorporated herein by reference in their entirety. This application is also related to the subject matter of commonly assigned, co-pending U.S. application Ser. No. 18/151,686 filed Jan. 9, 2023.

### BACKGROUND

### Technical Field

The present invention relates to T-cell receptors and, more particularly, to T-cell receptor (TCR) engineering with deep reinforcement learning for increasing efficacy and safety of TCR-T immunotherapy.

### Description of the Related Art

T cells monitor the health status of cells by identifying foreign peptides displayed on their surface. T-cell receptors (TCRs), which are protein complexes found on the surface of T cells, can bind to these peptides. This process is known as TCR recognition and constitutes a key step for immune response. Optimizing TCR sequences for TCR recognition represents a fundamental step towards the development of personalized treatments to trigger immune responses killing cancerous or virus-infected cells.

### SUMMARY

A method for implementing deep reinforcement learning with T-cell receptor (TCR) mutation policies to generate binding TCRs recognizing target peptides for immunotherapy is presented. The method includes extracting peptides to identify a virus or tumor cells, collecting a library of TCRs from target patients, predicting, by a deep neural network, interaction scores between the extracted peptides and the TCRs from the target patients, developing a deep reinforcement learning (DRL) framework with TCR mutation policies to generate TCRs with maximum binding scores, defining reward functions based on a reconstruction-based score and a density estimation-based score, randomly sampling batches of TCRs and following a policy network to mutate the TCRs, outputting mutated TCRs, ranking the outputted TCRs to utilize top-ranked TCR candidates to target the virus or the tumor cells for immunotherapy, and for each top-ranked TCR candidate, repeatedly identifying a set of self-peptides that the top-ranked TCR candidate binds to and further optimizing the top-ranked TCR candidate greedily by maximizing a sum of its interaction scores with a given set of peptide antigens while minimizing a sum of its interaction scores with the set of self-peptides until one or more stopping criteria of efficacy and safety are met.

A non-transitory computer-readable storage medium comprising a computer-readable program for implementing deep reinforcement learning with T-cell receptor (TCR) mutation policies to generate binding TCRs recognizing target peptides for immunotherapy is presented. The computer-readable program when executed on a computer causes the computer to perform the steps of extracting peptides to identify a virus or tumor cells, collecting a library of TCRs from target patients, predicting, by a deep neural network, interaction scores between the extracted peptides and the TCRs from the target patients, developing a deep reinforcement learning (DRL) framework with TCR mutation policies to generate TCRs with maximum binding scores, defining reward functions based on a reconstruction-based score and a density estimation-based score, randomly sampling batches of TCRs and following a policy network to mutate the TCRs, outputting mutated TCRs, ranking the outputted TCRs to utilize top-ranked TCR candidates to target the virus or the tumor cells for immunotherapy, and for each top-ranked TCR candidate, repeatedly identifying a set of self-peptides that the top-ranked TCR candidate binds to and further optimizing the top-ranked TCR candidate greedily by maximizing a sum of its interaction scores with a given set of peptide antigens while minimizing a sum of its interaction scores with the set of self-peptides until one or more stopping criteria of efficacy and safety are met.

A system for implementing deep reinforcement learning with T-cell receptor (TCR) mutation policies to generate binding TCRs recognizing target peptides for immunotherapy is presented. The system includes a memory and one or more processors in communication with the memory configured to extract peptides to identify a virus or tumor cells, collect a library of TCRs from target patients, predict, by a deep neural network, interaction scores between the extracted peptides and the TCRs from the target patients, develop a deep reinforcement learning (DRL) framework with TCR mutation policies to generate TCRs with maximum binding scores, define reward functions based on a reconstruction-based score and a density estimation-based score, randomly sample batches of TCRs and following a policy network to mutate the TCRs, output mutated TCRs, rank the outputted TCRs to utilize top-ranked TCR candidates to target the virus or the tumor cells for immunotherapy, and for each top-ranked TCR candidate, repeatedly identify a set of self-peptides that the top-ranked TCR candidate binds to and further optimize the top-ranked TCR candidate greedily by maximizing a sum of its interaction scores with a given set of peptide antigens while minimizing a sum of its interaction scores with the set of self-peptides until one or more stopping criteria of efficacy and safety are met.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block/flow diagram of an exemplary model architecture of the T-cell receptor proximal policy optimization (TCRPPO), in accordance with embodiments of the present invention;

FIG. 2 is block/flow diagram of exemplary data flow for the TCRPPO and T-cell receptor autoencoder (TCR-AE) training, in accordance with embodiments of the present invention;

FIG. 3 is a block/flow diagram of a practical application for T-cell receptor optimization with reinforcement learning and mutation policies for precision immunotherapy, in accordance with embodiments of the present invention;

FIG. 4 is an exemplary processing system for T-cell receptor optimization with reinforcement learning and mutation policies for precision immunotherapy, in accordance with embodiments of the present invention; and

FIG. 5 is a block/flow diagram of an exemplary method for T-cell receptor optimization with reinforcement learning and mutation policies for precision immunotherapy, in accordance with embodiments of the present invention.

### DETAILED DESCRIPTION OF PREFERRED EMBODIMENTS

Immunotherapy is a fundamental treatment for human diseases, which uses a person's immune system to fight diseases. In the immune system, immune response is triggered by cytotoxic T cells which are activated by the engagement of the T cell receptors (TCRs) with immunogenic peptides presented by Major Histocompatibility Complex (MHC) proteins on the surface of infected or cancerous cells. The recognition of these foreign peptides is determined by the interactions between the peptides and TCRs on the surface of T cells. This process is known as TCR recognition and constitutes a key step for immune response. Adoptive T cell immunotherapy (ACT), which has been a promising cancer treatment, genetically modifies the autologous T cells taken from patients in laboratory experiments, after which the modified T cells are infused into patients' bodies to fight cancer.

As one type of ACT therapy, TCR T cell (TCR-T) therapy directly modifies the TCRs of T cells to increase the binding affinities, which makes it possible to recognize and kill tumor cells effectively. TCR is a heterodimeric protein with an α chain and a β chain. Each chain has three loops as complementary determining regions (CDR): CDR1, CDR2 and CDR3. CDR1 and CDR2 are primarily responsible for interactions with MHC, and CDR3 interacts with peptides. The CDR3 of the β chain has a higher degree of variations and is therefore arguably mainly responsible for the recognition of foreign peptides. The exemplary embodiments focus on the optimization of the CDR3 sequence of β chain in TCRs to enhance their binding affinities against peptide antigens, and the optimization is conducted through reinforcement learning. The success of the exemplary approach will have the potential to guide TCR-T therapy design. For the sake of simplicity, when the exemplary methods refer to TCRs, it is meant the CDR3 of β chain in TCRs.

Despite the significant promise of TCR-T therapy, optimizing TCRs for therapeutic purposes remains a time-consuming process, which usually requires exhaustive screening for high-affinity TCRs, either in vitro or in silico. To accelerate this process, computational methods have been developed recently to predict peptide-TCR interactions, leveraging the experimental peptide-TCR binding data and TCR sequences. However, these peptide-TCR binding prediction tools cannot immediately direct the rational design of new high-affinity TCRs. Existing computational methods for biological sequence design include search-based methods, generative methods, optimization-based methods and reinforcement learning (RL)-based methods. However, all these methods generate sequences without considering additional conditions such as peptides, and thus cannot optimize TCRs tailored to recognizing different peptides. In addition, these methods do not consider the validity of generated sequences, which is important for TCR optimization as valid TCRs should follow specific characteristics.

The exemplary embodiments present a new reinforcement-learning (RL) framework based on proximal policy optimization (PPO), referred to as TCRPPO, to computationally optimize TCRs through a mutation policy. In particular, TCRPPO learns a joint policy to optimize TCRs customized for any given peptides. In TCRPPO, a new reward function is presented that measures both the likelihoods of the mutated sequences being valid TCRs, and the probabilities of the TCRs recognizing peptides.

In the reward design, besides maximizing a sum of the interaction scores between a mutated TCR under consideration and a set of given peptide antigens, the exemplary embodiments also minimize a sum of interaction scores between the mutated TCR under consideration and a set of self-peptides from normal human tissues that are most similar to the given set of peptide antigens. After the TCR optimization, the exemplary embodiments identify a set of self-peptides that the optimized TCR potentially binds to. To ensure the safety of TCR-T therapy, the exemplary embodiments further mutate the TCR to maximize the sum of the interaction scores between the mutated TCR under consideration and the set of given peptide antigens while minimizing the sum of the interaction scores between the TCR and the set of identified self-peptides. These steps are repeated until convergence or some specified immunotherapy safety control criteria is met.

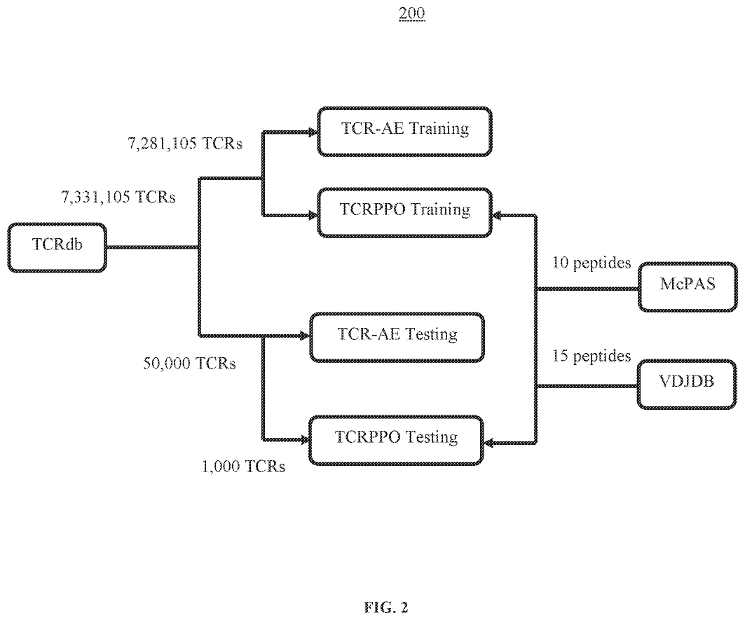

To measure TCR validity, a TCR auto-encoder was developed, referred to as TCR-AE, and reconstruction errors were utilized from TCR-AE and also its latent space distributions, quantified by a Gaussian Mixture Model GMM), to calculate novel validity scores. To measure peptide recognition, the exemplary methods leveraged a state-of-the-art peptide-TCR binding predictor ERGO to predict peptide-TCR binding. It is noted that TCRPPO is a flexible framework, as ERGO can be replaced by any other binding predictors. In addition, a novel buffering mechanism referred to as Buf-Opt is presented to revise TCRs that are difficult to optimize. Extensive experiments were conducted using 7 million TCRs from TCRdb 200 (FIG. 2), 10 peptides from McPAS and 15 peptides from VDJDB. The experimental results demonstrated that TCRPPO can substantially outperform the best baselines with best improvement of 58.2% and 26.8% in terms of generating qualified TCRs with high validity scores and high recognition probabilities, over McPAS and VDJDB peptides, respectively.

The recognition ability of a TCR sequence against the given peptides is measured by a recognition probability, denoted as sᵣ. The likelihood of a sequence being a valid TCR is measured by a score, denoted as 5, A qualified TCR is defined as a sequence with sᵣ>σᵣ, and sᵣ>σ_{c}, where σᵣ, and σ_{c}, are pre-defined thresholds. The goal of TCRPPO is to mutate the existing TCR sequences that have low recognition probability against the given peptide, into qualified ones. A peptide p or a TCR sequence c is represented as a sequence of its amino acids [symbol] o₁, o₂, . . . , oᵢ, . . . , oₗ [symbol] , where oᵢ is one of the 20 types of natural amino acids at the position i in the sequence, and l is the sequence length. The TCR mutation process was formulated as a Markov Decision Process (MDP) M={S, A, P, R} including the following components:

S: the state space, in which each state s∈S is a tuple of a potential TCR sequence c and a peptide p, that is, s=(c, p). Subscript t(t=0, . . . , T) is used to index step of s, that is, sₜ=(cₜ, p). It is noted that cₜ may not be a valid TCR. A state sₜ is a terminal state, denoted as S_{T}, if it includes a qualified cₜ, or t reaches the maximum step limit T It is also noted that p will be sampled at so and will not change over time t,

A: the action space, in which each action a∈A is a tuple of a mutation site i and a mutant amino acid o, that is, a=(i, o). Thus, the action will mutate the amino acid at position i of a sequence c=(o₁, o₂, . . . , oᵢ, . . . , oₗ) into another amino acid o. Note that o has to be different from oᵢ in c.

P: the state transition probabilities, in which P(sₜ₊₁|sₜ, aₜ) specifies the probability of next state sₜ₊₁ at time t+1 from state sₜ at time t with the action aₜ. In the problem, the transition to sₜ₊₁ is deterministic, that is P(sₜ₊₁|Sₜ, aₜ)=1.

R: the reward function at a state. In TCRPPO, all the intermediate rewards at states sₜ (t=0, . . . , T−1) are 0. Only the final reward at S_{T} is used to guide the optimization.

Regarding the mutation policy network, TCRPPO mutates one amino acid in a sequence c at a step to modify c into a qualified TCR. Specifically, at the initial step t=0, a peptide p is sampled as the target, and a valid TCR c₀ is sampled to initialize s₀=(c₀, p); at a state sₜ=(cₜ, p) (t>0), the mutation policy network of TCRPPO predicts an action at that mutates one amino acid of cₜ to modify it into cₜ₊₁ that is more likely to lead to a final, qualified TCR bound top. TCRPPO encodes the TCRs and peptides in a distributed embedding space. It then learns a mapping between the embedding space and the mutation policy, as discussed below.

Regarding encoding of amino acids, each amino acid o is represented by concatenating three vectors: o^{b}, the corresponding row of o in the BLOSUM matrix, o^{o}, the one-hot encoding of o, and o^{d}, the learnable embedding, that is, o is encoded as o=o^{b} ⊕o^{o}⊕o^{d}, where ⊕ represents the concatenation operation. The exemplary methods used such a mixture of encoding methods to enrich the representations of amino acids within c and p.

Regarding the embedding of states, sₜ=(cₜ, p) was embedded via embedding its associated sequences cₜ and p. For each amino acid o_{i,t} in cₜ, the exemplary methods embedded o_{i,t} and its context information in cₜ into a hidden vector h_{i,t} using a one-layer bidirectional long short-term memory (LSTM) as below:

\vec{h}_{i,t},\vec{c}_{i,t}=LSTM(o_{i,t},\vec{h}_{i,t},\vec{c}_{i-1,t};\vec{W});

[symbol]

_{i,t}, [symbol] _{i,t}=LSTM(o_{i,t}, [symbol] _{i,t}, [symbol] _{i,t}; [symbol] );

h_{i,t}=\vec{h}_{i,t}⊕ [symbol] _{i,t} (1)

where \vec{h}_{i,t} and [symbol] _{i,t} are the hidden state vectors of the i-th amino acid in cₜ;

\vec{c}_{i,t} and [symbol] _{i,t} are the memory cell states of i-th amino acid;

\vec{W} and [symbol] are the learnable parameters of the two LSTM directions, respectively; and

\vec{h}_{0,t}, [symbol] ₗ_{_{c}}_{,t}, \vec{c}_{0,t} and [symbol] ₗ_{_{c}}_{,t} (l_{c} is the length of cₜ) are initialized with random vectors. With the embeddings of all the amino acids, the embedding of cₜ were defined as the concatenation of hidden vectors at the two ends, that is, hₜ=\vec{h}ₗ_{_{c}}_{,t}⊕ [symbol] _{0,t}.

A peptide sequence was embedded into a hidden vector h^{p} using another bidirectional LSTM in the same way.

Regarding action prediction, to predict the action aₜ=(i, o) at time t, TCRPPO needs to make two predictions, that is, the position i of current cₜ where at needs to occur and the new amino acid o that aₜ needs to place with at position i. To measure “how likely” the position i in cₜ is the action site, TCRPPO uses the following network:

f(i)=w^{T}(ReLU(W₁h_{i,t}+W₂h^{p}))/(Σⱼ₌₁^{l}^{_{c}}w^{T}(ReLU(W₁h_{i,t}+W₂h^{p}))), (2)

where h_{i,t} the latent vector of o_{i,t} in cₜ (Equation 1); h^{p} is the latent vector of p; and w/Wⱼ (j=1, 2) are the learnable vector/matrices. Thus, TCRPPO measures the probability of position i being the action site by looking at its context encoded in h_{i,t} and the peptide p. The predicted position i is sampled from the probability distribution from Equation 2 to ensure necessary exploration.

Given the predicted position i, TCRPPO needs to predict the new amino acid that should replace oᵢ in cₜ. TCRPPO calculates the probability of each amino acid type being the new replacement as follows:

g(o)=softmax(U₁×ReLU(U₂h_{i,t}+U₃h^{p})), (3)

where Uⱼ (j=1,2,3) are the learnable matrices; and softmax(·) converts a 20-dimensional vector into probabilities over the 20 amino acid types. The replacement amino acid type is then determined by sampling from the distribution, excluding the original type of o_{i,t}.

Regarding potential TCR validity measurement, a novel scoring function is presented to quantitatively measure the likelihood of a given sequence c being a valid TCR (e.g., to calculate sᵥ), which will be part of the reward of TCRPPO. Specifically, the exemplary methods trained a novel auto-encoder model, denoted as TCR-AE, from only valid TCRs. The reconstruction accuracy of a sequence in TCR-AE was used to measure its TCR validity. The intuition is that since TCR-AE is trained from only valid TCRs, its encoding-decoding process will obey the “rules” of true TCR sequences, and thus, a non-TCR sequence could not be well reproduced from TCR-AE. However, it is still possible that a non-TCR sequence can receive a high reconstruction accuracy from TCR-AE, if TCR-AE learns some generic patterns shared by TCRs and non-TCRs and fails to detect irregularities, or TCR-AE has high model complexity. To mitigate this, the exemplary methods additionally evaluate the latent space within TCR-AE using a Gaussian Mixture Model (GMM), hypothesizing that non-TCRs would deviate from the dense regions of TCRs in the latent space.

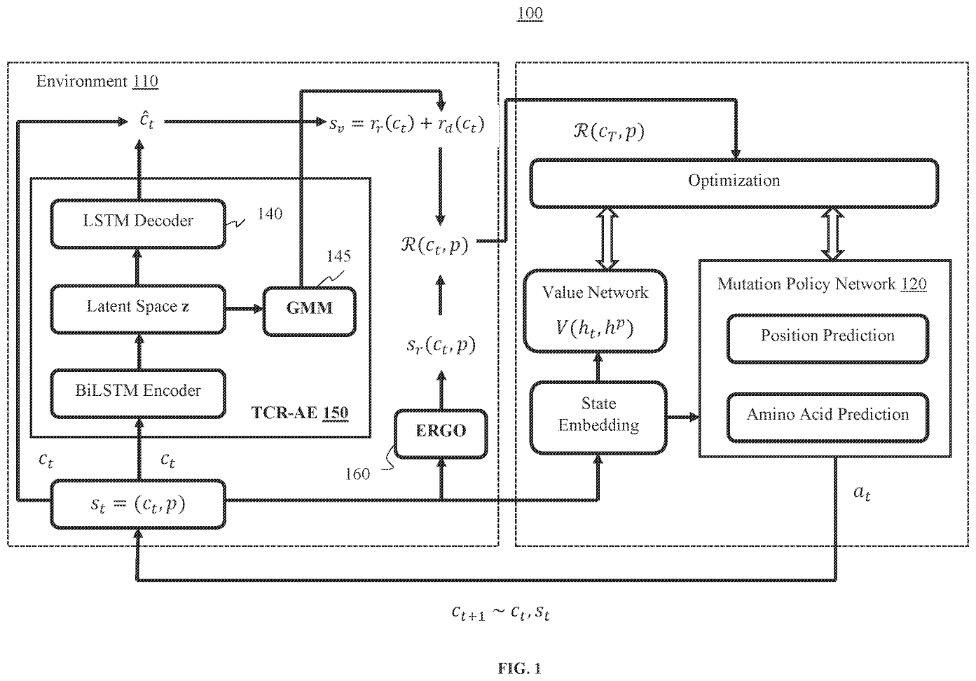

TCR-AE 150, as shown in the TCRPPO 100 of FIG. 1, presents the auto-encoder TCR-AE. TCR-AE 150 uses a bidirectional LSTM to encode an input sequence c into h′ by concatenating the last hidden vectors from the two LSTM directions (similarly as in Equation 1). h′ is then mapped into a latent embedding z′ as follows,

z′=W^{z}h′, (4)

which will be decoded back to a sequence ĉ via a decoder 140. The decoder 140 has a single-directional LSTM that decodes z′ by generating one amino acid at a time as follows,

h′ᵢ,c′ᵢ=LSTM(ôᵢ₋₁,h′ᵢ₋₁,c′ᵢ₋₁;W′);ôᵢ=softmax(U′×ReLU(U′₁h′ᵢ+U′₂z′)), (5)

where ôᵢ₋₁ is the encoding of the amino acid ôᵢ₋₁ that is decoded from step i−1; and W′ is the parameter. The LSTM starts with a zero vector o₀=0 and h₀=W^{hz′}. The decoder infers the next amino acid by looking at the previously decoded amino acids encoded in h′ᵢ and the entire prospective sequence encoded in z′.

It is noted that TCR-AE 150 is trained from TCRs, independently of TCRPPO 100 and in an end-to-end fashion. Teacher forcing is applied during training to ensure that the decoded sequence has the same length as the input sequence, and thus, cross entropy loss is applied to optimize TCR-AE 150. As a stand-alone module, TCR-AE 150 is used to calculate the score sᵥ. The input sequence c to TCR-AE 150 is encoded using only the BLOSUM matrix as it is found empirically that BLOSUM encoding can lead to a good reconstruction performance and a fast convergence compared to other combinations of encoding methods.

With a well-trained TCR-AE 150, the reconstruction-based TCR validity score of a sequence c was calculated as follows,

rᵣ(c)=1−lev(c,TCR-AE(c))/l_{c} (6)

where TCR-AE(c) represents the reconstructed sequence of c from TCR-AE; lev(c, TCR-AE(c)) is the Levenshtein distance, an edit-distance-based metric, between c and TCR-AE(c); l_{c} is the length of c. Higher rᵣ(c) indicates higher probability of c being a valid TCR. It is noted that when TCR-AE 150 is used in testing, the length of the reconstructed sequence might not be the same as the input c, because TCR-AE 150 could fail to accurately predict the end of the sequence, leading to either too short or too long reconstructed sequences. Therefore, the Levenshtein distance is normalized using the length of input sequence l_{c}. It is noted that rᵣ(c) could be negative when the distance is greater than the sequence length. The negative values will not affect the use of the scores (e.g., negative rᵣ(c) indicates very different TCR-AE(c) and c).

To better distinguish valid TCRs from invalid ones, TCRPPO 100 also conducts a density estimation over the latent space of z′ (Equation 4) using GMM 145.

For a given sequence c, TCRPPO 100 calculates the likelihood score of c falling within the Gaussian mixture region of training TCRs as follows,

$${r}_{d} ( c ) = exp ( 1 + \frac{log P ( {z}^{′} )}{τ} ) \qquad ( 7 )$$

where log P(z′) is the log-likelihood of the latent embedding z; and τ is a constant used to rescale the log-likelihood value (τ=10). The parameter τ is carefully selected such that 90% of TCRs can have r_{d}(c) above 0.5. Since no invalid TCRs are had, the exemplary methods cannot use classification-based scaling methods such as Platt scaling to calibrate the log likelihood values to probabilities.

Combining the reconstruction-based scoring and density estimation-based scoring, a new scoring method was developed to measure TCR validity as follows:

sᵥ(c)=rᵣ(c)+r_{d}(c). (8)

This method is used to evaluate if a sequence is likely to be a valid TCR and is used in the reward function.

Regarding TCRPPO learning, and with respect to the final reward, the exemplary methods defined the final reward for TCRPPO 100 based on sᵣ and sᵥ scores as follows,

[symbol]

(c_{T},p)=sᵣ(c_{T},p)+α min(0,sᵥ(c_{T})−σc) (9)

where sᵣ(c_{T}, p) is the predicted recognition probability by ERGO 160, σ_{c} is a threshold that c_{T} is very likely to be a valid TCR (σ_{c}=1.2577); and a is the hyperparameter used to control the tradeoff between sᵣ and sᵥ(α=0.5).

Regarding policy learning, the exemplary methods adopt the proximal policy optimization (PPO) to optimize the policy network.

The objective function of PPO is defined as follows:

$${max}_{Θ} {L}^{CLIP} ( Θ ) = {\hat{𝔼}}_{t} [ min ( {r}_{t} ( Θ ) {\hat{A}}_{t} , clip ( {r}_{t} ( Θ ) , 1 - ϵ , 1 + ϵ ) {\hat{A}}_{t} ) ] ,$$

$$where {r}_{t} ( Θ ) = \frac{{π}_{Θ} ( {a}_{t} \vert {s}_{t} )}{{π}_{{Θ}_{old}} ( {a}_{t} \vert {s}_{t} )} ,$$

where Θ is the set of learnable parameters of the policy network and rₜ(Θ) is the probability ratio between the action under current policy πΘ and the action under previous policy πΘ_{old}. Here, rₜ(Θ) is clipped to avoid moving rₜ outside of the interval [1−ϵ, 1+ϵ].

Âₜ is the advantage at timestep t computed with the generalized advantage estimator, measuring how much better a selected action is than others on average:

Âₜ=δₜ+(γλ)δₜ₊₁+ . . . +(γλ)^{t-t+1}δ_{T+1},

where γ∈(0, 1) is the discount factor determining the importance of future rewards; δₜ=rₜ+γV (sₜ₊₁)−V(Sₜ) is the temporal difference error in which V (Sₜ) is a value function; and λ∈ (0, 1) is a parameter used to balance the bias and variance of V (Sₜ). V(·) uses a multi-layer perceptron (MLP) to predict the future return of current state sₜ from the peptide embedding h^{p} and the TCR embedding hₜ.

The objective function of V(·) is as follows:

min_{Θ}L^{V}(Θ)= [symbol] [(V(hₜ,h^{p})−\hat{R}ₜ)²],

where \hat{R}ₜ=Σᵢ₌ₜ₊₁^{T}γ^{i-t}rᵢ is the rewards-to-go. Because only the final rewards are used, that is rᵢ=0 if i≠T, the exemplary methods calculated \hat{R}ₜ with \hat{R}ₜ=γ^{T-t}r_{T}. The entropy regularization loss H(Θ) was also added, a popular strategy used for policy gradient methods to encourage the exploration of the policy.

The final objective function of TCRPPO 100 is defined as below,

min_{Θ}L(Θ)=−L^{CLIP}(Θ)+α₁L^{V}(Θ)−α₂H(Θ),

where α₁ and α₂ are two hyperparameters controlling the tradeoff among the PPO objective, the value function and the entropy regularization term.

TCRPPO 100 implements a novel buffering and re-optimizing mechanism, denoted as Buf-Opt, to deal with TCRs that are difficult to optimize, and to generalize its optimization capacity to more diverse TCRs. This mechanism includes a buffer, which memorizes the TCRs that cannot be optimized to qualify. These hard sequences will be sampled from the buffer again following the probability distribution below, to be further optimized by TCRPPO 100,

S(c,p)= [symbol] (10)

In Equation 10, S measures how difficult it is to optimize c against p based on its final reward R(c_{T}, p) in the previous optimization, ξ is hyper-parameter (e.g., ξ=5), and Σ converts S(c, p) as a probability. It is expected that by doing the sampling and re-optimization, TCRPPO 100 is better trained to learn from hard sequences, and also the hard sequences have the opportunity to be better optimized by TCRPPO 100. In case a hard sequence still cannot be optimized to qualify, it will have a 50% chance of being allocated back to the buffer. In case the buffer is full (size 2,000 in experiments), the sequences earliest allocated in the buffer will be removed. The TCRPPO 100 with Buf-Opt is referred to as TCRPPO+b.

In conclusion, the exemplary embodiments of the present invention formulated the search for optimized TCRs as a RL problem and presented a framework TCRPPO with a mutation policy using proximal policy optimization (PPO). TCRPPO mutates TCRs into effective ones that can recognize given peptides. TCRPPO leverages a reward function that combines the likelihoods of mutated sequences being valid TCRs measured by a new scoring function based on deep autoencoders, with the probabilities of mutated sequences recognizing peptides from a peptide-TCR interaction predictor. TCRPPO was compared with multiple baseline methods and demonstrated that TCRPPO significantly outperforms all the baseline methods to generate positive binding and valid TCRs. These results demonstrate the potential of TCRPPO for both precision immunotherapy and peptide recognizing TCR motif discovery.

The exemplary methods further present a deep reinforcement learning system with TCR mutation policies for generating binding TCRs recognizing target peptides. The pre-defined library of peptides can be derived from the genome of a virus such as SARS-CoV-2 or from sequencing tumor samples of a patient. Therefore, the presented exemplary system can be used for immunotherapy targeting a particular type of virus or tumor with TCR engineering.

Given a virus genome or some tumor cells, the exemplary methods run sequencing followed by some off-the-shelf peptide processing pipelines to extract some peptides that can uniquely identify the virus or tumor cells. The exemplary methods also collect a library of TCRs from target patients. Targeting this peptide library from the virus or tumor and the given TCRs, the system can generate optimized TCRs or mutated TCRs so that immune responses can be triggered to kill the virus or tumor cells.

The exemplary methods first train a deep neural network on the public IEDB, VDJdb, and McPAS-TCR datasets or a pre-trained model such as ERGO is downloaded to predict the binding interaction between peptides and TCRs. Based on this pre-trained model for predicting peptide-TCR interaction scores, the exemplary methods develop a DRL system with TCR mutation policies to generate TCRs with high binding scores that are the same as or at most d amino acids different from the provided library of TCRs. Specifically, using the pretrained prediction deep model to define reward functions and starting from random or existing TCRs, the exemplary methods then pretrain a DRL system to learn good TCR mutation policies transforming a given random TCR into a peptide recognizing TCR with a high binding interaction score. Based on this trained DRL system with pretrained TCR mutation policies, the exemplary methods randomly sample batches of TCRs from the provided library and follow the policy network to mutate the TCRs. During the mutation process, if any mutated TCR is already d amino acid different from the starting TCR, the process is topped and the TCR is output as final TCR. The final mutated TCRs recognizing given peptides are outputted and the compiled set of mutated TCRs are ranked. The top ranked ones will be used as promising engineered TCRs targeting the specified virus or tumor cells for immunotherapy.

In the reward design, besides maximizing a sum of the interaction scores between a mutated TCR under consideration and a set of given peptide antigens, the exemplary embodiments also minimize a sum of interaction scores between the mutated TCR under consideration and a set of self-peptides from normal human tissues that are most similar to the given set of peptide antigens. After the TCR optimization, the exemplary embodiments identify a set of self-peptides that the optimized TCR potentially binds to. To ensure the safety of TCR-T therapy, the exemplary embodiments further mutate the TCR to maximize the sum of the interaction scores between the mutated TCR under consideration and the set of given peptide antigens while minimizing the sum of the interaction scores between the TCR and the set of identified self-peptides. These steps are repeated until convergence or some specified immunotherapy safety control criteria is met.

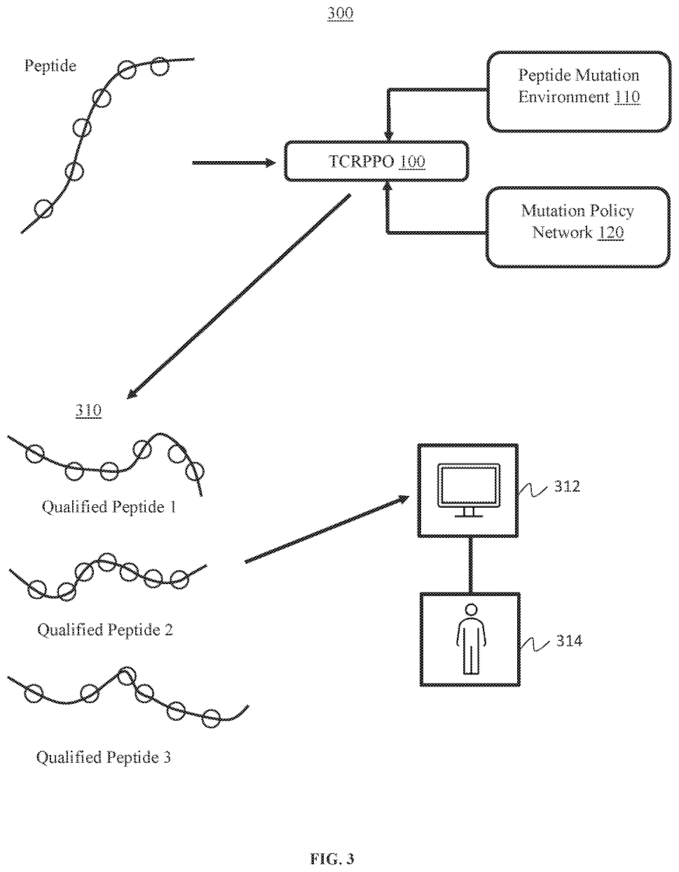

FIG. 3 is an exemplary practical application for T-cell receptor optimization with reinforcement learning and mutation policies for precision immunotherapy, in accordance with embodiments of the present invention.

In one practical example 300, a peptide is processed by the TCRPPO 100 within the peptide mutation environment 110 by the mutation policy network 120 to generate new qualified peptides 310 to be displayed on a screen 312 and analyzed by a user 314. For all the selected peptides from a same database (e.g., 10 peptides from McPAS, 15 peptides from VDJDB), the exemplary methods trained one TCRPPO agent, which optimizes the training sequences (e.g., 7,281,105 TCRs in FIG. 2) to be qualified against one of the selected peptides. The ERGO model trained on the corresponding database will be used to test recognition probabilities sᵣ for the TCRPPO agent. It is noted that one ERGO model is trained for all the peptides in each database (e.g., one ERGO predicts TCR-peptide binding for multiple peptides). Thus, the ERGO model is suitable to test sᵣ for multiple peptides in the exemplary setting. Also, it is noted that the exemplary methods trained one TCRPPO agent corresponding to each database, because peptides and TCRs in these two databases are very different, demonstrated by the inferior performance of an ERGO trained over the two databases together.

TCRPPO mutates each sequence up to 8 steps (T=8), which is large enough as the most popular length of TCRs is 15. In TCRPPO training (FIG. 2), an initial TCR sequence (e.g., c₀ in so) is randomly sampled from Sₜᵣₙ, and is mutated in the following states: a peptide p is randomly sampled at so and remains the same in the following states (e.g., sₜ=(cₜ, p)). Once the TCRPPO 100 is well trained from Sₜᵣₙ, it will be tested on Stat.

The experimental results in comparison with generation-based methods and mutation-based methods on optimizing TCRs demonstrate that TCRPPO 100 significantly outperforms the baseline methods. The analysis on the TCRs generated by TCRPPO 100 demonstrates that TCRPPO 100 can successfully learn the conservation patterns of TCRs. The experiments on the comparison between the generated TCRs and existing TCRs demonstrate that TCRPPO 100 can generate TCRs similar to existing human TCRs, which can be used for further medical evaluation and investigation. The results in TCR detection comparison show that the sᵥ score in the exemplary framework can very effectively detect non-TCR sequences. The analysis on the distribution of sᵥ scores over mutations demonstrates that TCRPPO 100 mutates sequences along the trajectories not far away from valid TCRs.

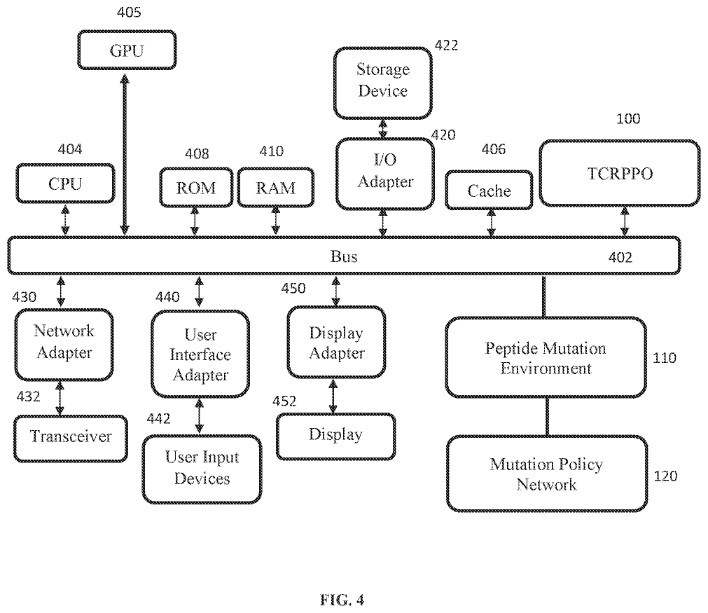

FIG. 4 is an exemplary processing system for T-cell receptor optimization with reinforcement learning and mutation policies for precision immunotherapy, in accordance with embodiments of the present invention.

The processing system includes at least one processor (CPU) 404 operatively coupled to other components via a system bus 402. A Graphical Processing Unit (GPU) 405, a cache 406, a Read Only Memory (ROM) 408, a Random Access Memory (RAM) 410, an Input/Output (I/O) adapter 420, a network adapter 430, a user interface adapter 440, and a display adapter 450, are operatively coupled to the system bus 402. Additionally, the TCRPPO 100 is employed within the peptide mutation environment 110 by using the mutation policy network 120.

A storage device 422 is operatively coupled to system bus 402 by the I/O adapter 420. The storage device 422 can be any of a disk storage device (e.g., a magnetic or optical disk storage device), a solid-state magnetic device, and so forth.

A transceiver 432 is operatively coupled to system bus 402 by network adapter 430.

User input devices 442 are operatively coupled to system bus 402 by user interface adapter 440. The user input devices 442 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present invention. The user input devices 442 can be the same type of user input device or different types of user input devices. The user input devices 442 are used to input and output information to and from the processing system.

A display device 452 is operatively coupled to system bus 402 by display adapter 450.

Of course, the processing system may also include other elements (not shown), as readily contemplated by one of skill in the art, as well as omit certain elements. For example, various other input devices and/or output devices can be included in the system, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system are readily contemplated by one of ordinary skill in the art given the teachings of the present invention provided herein.

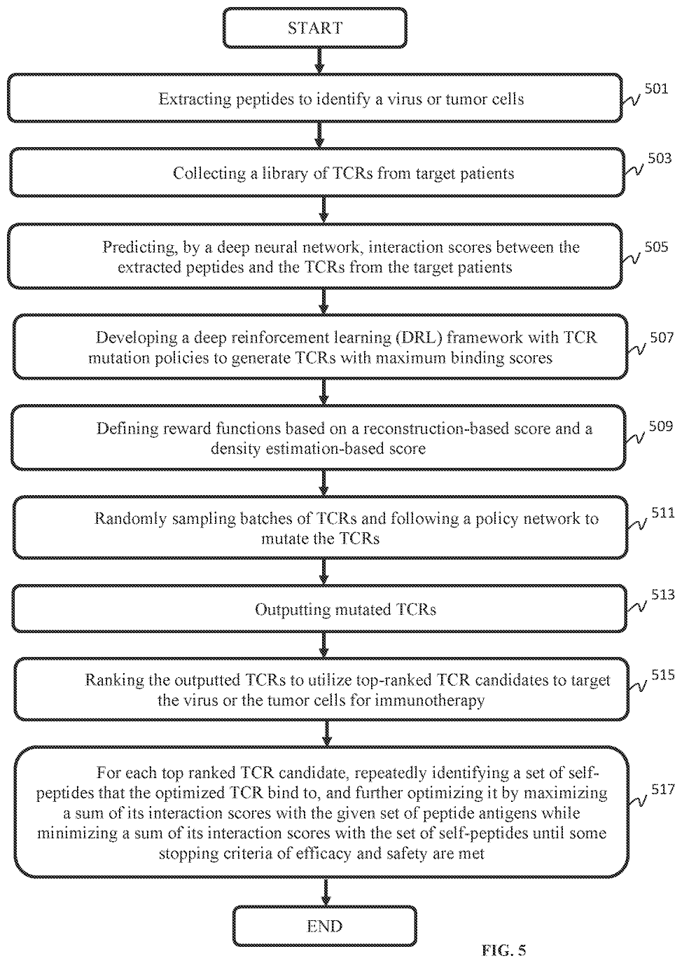

FIG. 5 is a block/flow diagram of an exemplary method for T-cell receptor optimization with reinforcement learning and mutation policies for precision immunotherapy, in accordance with embodiments of the present invention.

At block 501, extract peptides to identify a virus or tumor cells.

At block 503, collect a library of TCRs from target patients.

At block 505, predict, by a deep neural network, interaction scores between the extracted peptides and the TCRs from the target patients.

At block 507, develop a deep reinforcement learning (DRL) framework with TCR mutation policies to generate TCRs with maximum binding scores.

At block 509, define reward functions based on a reconstruction-based score and a density estimation-based score.

At block 511, randomly sample batches of TCRs and following a policy network to mutate the TCRs.

At block 513, output mutated TCRs.

At block 515, rank the outputted TCRs to utilize top-ranked TCR candidates to target the virus or the tumor cells for immunotherapy.

At block 517, for each top-ranked TCR candidate, repeatedly identify a set of self-peptides that the top-ranked TCR candidate binds to and further optimize the top-ranked TCR candidate by maximizing a sum of its interaction scores with a given set of peptide antigens while minimizing a sum of its interaction scores with the set of self-peptides until one or more stopping criteria of efficacy and safety are met.

In conclusion, the exemplary methods propose a DRL system with TCR mutation policies for generating binding TCRs recognizing given peptide antigens. The presented system can be used for generating TCRs for immunotherapy targeting a particular type of virus or tumor. The reward design is based on a TCR in-distribution score and the binding interaction score. The exemplary methods use PPO to optimize the DRL model and output the final mutated TCRs and rank the compiled set of mutated TCRs. The top ranked ones will be used as promising candidates targeting the specified virus or tumor for immunotherapy. For each top ranked TCR, a set of self-peptides that the optimized TCR potentially binds to are identified. To ensure the safety of TCR-T therapy, the TCR is mutated to maximize the sum of the interaction scores between the mutated TCR under consideration and the set of given peptide antigens while minimizing the sum of the interaction scores between the TCR and the set of identified self-peptides (such steps are repeated until convergence or some stopping criteria is met and output the final set of optimized TCRs).

As used herein, the terms “data,” “content,” “information” and similar terms can be used interchangeably to refer to data capable of being captured, transmitted, received, displayed and/or stored in accordance with various example embodiments. Thus, use of any such terms should not be taken to limit the spirit and scope of the disclosure. Further, where a computing device is described herein to receive data from another computing device, the data can be received directly from another computing device or can be received indirectly via one or more intermediary computing devices, such as, for example, one or more servers, relays, routers, network access points, base stations, and/or the like.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” “calculator,” “device,” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, RAM, ROM, an Erasable Programmable Read-Only Memory (EPROM or Flash memory), an optical fiber, a portable CD-ROM, an optical data storage device, a magnetic data storage device, or any suitable combination of the foregoing. In the context of this document, a computer readable storage medium may be any tangible medium that can include, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or a WAN, or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the present invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks or modules.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks or modules.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks or modules.

It is to be appreciated that the term “processor” as used herein is intended to include any processing device, such as, for example, one that includes a CPU and/or other processing circuitry. It is also to be understood that the term “processor” may refer to more than one processing device and that various elements associated with a processing device may be shared by other processing devices.

The term “memory” as used herein is intended to include memory associated with a processor or CPU, such as, for example, RAM, ROM, a fixed memory device (e.g., hard drive), a removable memory device (e.g., diskette), flash memory, etc. Such memory may be considered a computer readable storage medium.

In addition, the phrase “input/output devices” or “I/O devices” as used herein is intended to include, for example, one or more input devices (e.g., keyboard, mouse, scanner, etc.) for entering data to the processing unit, and/or one or more output devices (e.g., speaker, display, printer, etc.) for presenting results associated with the processing unit.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A method for implementing deep reinforcement learning with T-cell receptor (TCR) mutation policies to generate binding TCRs recognizing target peptides for immunotherapy, the method comprising:
extracting peptides to identify a virus or tumor cells;
collecting a library of TCRs from target patients;
predicting, by a deep neural network, interaction scores between the extracted peptides and the TCRs from the target patients;
developing a deep reinforcement learning (DRL) framework with TCR mutation policies to generate TCRs with maximum binding scores;
defining reward functions based on a reconstruction-based score and a density estimation-based score;
randomly sampling batches of TCRs and following a policy network to mutate the TCRs;
outputting mutated TCRs;
ranking the outputted TCRs to utilize top-ranked TCR candidates to target the virus or the tumor cells for immunotherapy; and
for each top-ranked TCR candidate, repeatedly identifying a set of self-peptides that the top-ranked TCR candidate binds to and further optimizing the top-ranked TCR candidate greedily by maximizing a sum of its interaction scores with a given set of peptide antigens while minimizing a sum of its interaction scores with the set of self-peptides until one or more stopping criteria of efficacy and safety are met.

2. The method of claim 1, wherein the reward functions measure both a likelihood of mutated sequences being valid TCRs and probabilities of the TCRs recognizing peptides.

3. The method of claim 2, wherein the measurement of the likelihood of the mutated sequences being valid TCRs is enabled by a TCR autoencoder (TCR-AE) trained only by TCRs.

4. The method of claim 3, wherein density estimation over a latent space within the TCR-AE is evaluated by using a Gaussian Mixture Model (GMM).

5. The method of claim 3, wherein the TCR-AE uses a bidirectional long short-term memory (LSTM) to encode an input sequence into a hidden vector by concatenating last hidden vectors from two LSTM directions.

6. The method of claim 1, wherein a buffering and re-optimizing framework including a buffer is employed to handle TCRs difficult to optimize and to generalize optimization capacity to more diverse TCRs.

7. The method of claim 1, wherein the TCRs and the extracted peptides are encoded by a TCR-AE in a distributed embedding space, and a mapping is learnt between the embedding space and the TCR mutation policies.

8. A non-transitory computer-readable storage medium comprising a computer-readable program for implementing deep reinforcement learning with T-cell receptor (TCR) mutation policies to generate binding TCRs recognizing target peptides for immunotherapy, wherein the computer-readable program when executed on a computer causes the computer to perform the steps of:
extracting peptides to identify a virus or tumor cells;
collecting a library of TCRs from target patients;
predicting, by a deep neural network, interaction scores between the extracted peptides and the TCRs from the target patients;
developing a deep reinforcement learning (DRL) framework with TCR mutation policies to generate TCRs with maximum binding scores;
defining reward functions based on a reconstruction-based score and a density estimation-based score;
randomly sampling batches of TCRs and following a policy network to mutate the TCRs;
outputting mutated TCRs;
ranking the outputted TCRs to utilize top-ranked TCR candidates to target the virus or the tumor cells for immunotherapy; and
for each top-ranked TCR candidate, repeatedly identifying a set of self-peptides that the top-ranked TCR candidate binds to and further optimizing the top-ranked TCR candidate greedily by maximizing a sum of its interaction scores with a given set of peptide antigens while minimizing a sum of its interaction scores with the set of self-peptides until one or more stopping criteria of efficacy and safety are met.

9. The non-transitory computer-readable storage medium of claim 8, wherein the reward functions measure both a likelihood of mutated sequences being valid TCRs and probabilities of the TCRs recognizing peptides.

10. The non-transitory computer-readable storage medium of claim 9, wherein the measurement of the likelihood of the mutated sequences being valid TCRs is enabled by a TCR autoencoder (TCR-AE) trained only by TCRs.

11. The non-transitory computer-readable storage medium of claim 10, wherein density estimation over a latent space within the TCR-AE is evaluated by using a Gaussian Mixture Model (GMM).

12. The non-transitory computer-readable storage medium of claim 10, wherein the TCR-AE uses a bidirectional long short-term memory (LSTM) to encode an input sequence into a hidden vector by concatenating last hidden vectors from two LSTM directions.

13. The non-transitory computer-readable storage medium of claim 8, wherein a buffering and re-optimizing framework including a buffer is employed to handle TCRs difficult to optimize and to generalize optimization capacity to more diverse TCRs.

14. The non-transitory computer-readable storage medium of claim 8, wherein the TCRs and the extracted peptides are encoded by a TCR-AE in a distributed embedding space, and a mapping is learnt between the embedding space and the TCR mutation policies.

15. A system for implementing deep reinforcement learning with T-cell receptor (TCR) mutation policies to generate binding TCRs recognizing target peptides for immunotherapy, the system comprising:
a memory; and
one or more processors in communication with the memory configured to:
extract peptides to identify a virus or tumor cells;
collect a library of TCRs from target patients;
predict, by a deep neural network, interaction scores between the extracted peptides and the TCRs from the target patients;
develop a deep reinforcement learning (DRL) framework with TCR mutation policies to generate TCRs with maximum binding scores;
define reward functions based on a reconstruction-based score and a density estimation-based score;
randomly sample batches of TCRs and following a policy network to mutate the TCRs;
output mutated TCRs;
rank the outputted TCRs to utilize top-ranked TCR candidates to target the virus or the tumor cells for immunotherapy; and
for each top-ranked TCR candidate, repeatedly identify a set of self-peptides that the top-ranked TCR candidate binds to and further optimize the top-ranked TCR candidate greedily by maximizing a sum of its interaction scores with a given set of peptide antigens while minimizing a sum of its interaction scores with the set of self-peptides until one or more stopping criteria of efficacy and safety are met.

16. The system of claim 15, wherein the reward functions measure both a likelihood of mutated sequences being valid TCRs and probabilities of the TCRs recognizing peptides.

17. The system of claim 16, wherein the measurement of the likelihood of the mutated sequences being valid TCRs is enabled by a TCR autoencoder (TCR-AE) trained only by TCRs.

18. The system of claim 17, wherein density estimation over a latent space within the TCR-AE is evaluated by using a Gaussian Mixture Model (GMM).

19. The system of claim 17, wherein the TCR-AE uses a bidirectional long short-term memory (LSTM) to encode an input sequence into a hidden vector by concatenating last hidden vectors from two LSTM directions.

20. The system of claim 15, wherein a buffering and re-optimizing framework including a buffer is employed to handle TCRs difficult to optimize and to generalize optimization capacity to more diverse TCRs.
